(19) United States
(12) Patent Application Publication
(10) Pub. No.: US 2024/0358895 A1
(43) Pub. Date: Oct. 31, 2024

(54) MODEL TRAINING METHOD, VISUAL PERCEPTION METHOD, ELECTRONIC DEVICE AND STORAGE MEDIUM

(71) Applicant: Westlake University, Hangzhou (CN)

(72) Inventors: Chuanqing WANG, Hangzhou (CN); Jie YANG, Hangzhou (CN); MOHAMAD SAWAN, Hangzhou (CN)

(21) Appl. No.: 18/648,289

(22) Filed: Apr. 26, 2024

(30) Foreign Application Priority Data
Apr. 28, 2023 (CN) 202310486675.0

(51) Int. Cl. A61L 27/36; G06V 10/774; G06V 20/40

(52) U.S. Cl. CPC A61L 27/3695; A61L 27/3604; A61L 27/3675; G06V 10/774; G06V 20/46; A61L 2430/16

(57) ABSTRACT

Disclosed are a model training method, a visual perception method, an electronic device and a storage medium. The model training method is applicable to a spiking recurrent model in a retinal prosthesis and includes: determining labels respectively corresponding to ganglion cells based on a preset ganglion cell response dataset; obtaining multiple spike signals as training samples; inputting the spike signals into the spiking recurrent model, obtaining spike responses of the ganglion cells predicted by the spiking recurrent model, and computing a loss value based on the spike responses of the ganglion cells, the labels and a preset Poisson loss function; and updating a weight for each layer in the spiking recurrent model based on the loss value and a preset time backpropagation function recursively until the spiking recurrent model converges, thereby scientifically and quickly training the spiking recurrent model for predicting the responses of retinal ganglion cells.

### CROSS REFERENCE TO RELATED APPLICATIONS

The present application claims the benefit of priority under the Paris Convention to Chinese Patent Application No. 202310486675.0 filed on Apr. 28, 2023, which is incorporated herein by reference in its entirety.

### TECHNICAL FIELD

Embodiments of the present disclosure relate to the technical field of deep learning, and in particular, to a model training method, a visual perception method, an electronic device and a storage medium.

### BACKGROUND

Optogenetics-based retinal prosthesis are used to treat two visual diseases: age-related macular degeneration (AMD) and retinitis pigmentosa (RP). More than 100 million people worldwide suffer from these two types of visual degenerative diseases. As shown in FIG. 1, AMD and RP patients partially loss visual perception due to the degeneration of a large amount of cone and rod cells in the retina. The differences between the two diseases are the location of the degenerated cone and rod cells, as well as the order of cell apoptosis. At present, existing drug and gene therapy methods have limited effects on severe AMD and RP patients. With the development of optogenetics, retinal prosthesis based on optogenetics have become an important treatment for these two diseases.

The cone and rod cells on a normal functioning retina can effectively perceive external images and generate electroneurographic signals to encode these images. Bipolar cells and other cell layers process corresponding electroneurographic signals and then transmit the processed neural code to the cerebral cortex. A retinal prosthesis uses a model for replacing the signal processing capability of a retina to perceive and process images and then transmit processed signals through light stimulation to ganglion cells in the last layer of the retina, thereby forming visual perception in the visual cortex.

However, inventors of the present disclosure have found that current retinal prosthesis either can only simplify images and cannot replace the functions of cone and rod cells, or can predict the responses of retinal ganglion cells to replace the functions of cone and rod cells by using a convolutional neural network. However, the convolutional neural network uses floating-point multiplication for processing, which requires a large amount of computation, consumes a lot of energy and also lacks biological similarity, thus being unsuitable for use in retinal prosthesis.

### SUMMARY

Embodiments of the present disclosure are intended to provide a model training method, a visual perception method, an electronic device and a storage medium. The model training method can scientifically and quickly train a spiking recurrent model for predicting the responses of retinal ganglion cells. The trained spiking recurrent model has low power consumption at work, high prediction accuracy, and high biological similarity, effectively improving the visual perception ability of a retinal prosthesis.

To solve the above technical problem, embodiments of the present disclosure provide a model training method applicable to a spiking recurrent model in a retinal prosthesis. The spiking recurrent model is used for predicting spike responses of ganglion cells. The model training method includes: determining labels respectively corresponding to the ganglion cells based on a preset ganglion cell response dataset; obtaining a plurality of spike signals as training samples; inputting the spike signals into the spiking recurrent model, obtaining spike responses of the ganglion cells predicted by the spiking recurrent model, and computing a loss value based on the spike responses of the ganglion cells, the labels and a preset Poisson loss function; and updating a weight for each layer in the spiking recurrent model based on the loss value and a preset time backpropagation function recursively until the spiking recurrent model converges.

Embodiments of the present disclosure further provide a visual perception method applicable to a retinal prosthesis. The aforementioned spiking recurrent model is provided in the retinal prosthesis, and is configured for predicting spike responses of ganglion cells. The visual perception method includes: recording a target video and encoding the target video as real spike signals; inputting the real spike signals into the spiking recurrent model, and obtaining the spike responses of the ganglion cells generated by the spiking recurrent model; and transmitting the spike responses of the ganglion cells through light stimulation to the ganglion cells in the last layer of the retina of an implant recipient of the retinal prosthesis for visual perception.

Embodiments of the present disclosure further provide an electronic device, which includes: at least one processor; and a memory communicably connected with the at least one processor for storing instructions executable by the at least one processor. Execution of the instructions by the at least one processor causes the at least one processor to implement the aforementioned model training method, or to implement the aforementioned visual perception method.

Embodiments of the present disclosure further provide a computer-readable storage medium on which a computer program is stored. The computer program is executed by a processor to implement the aforementioned model training method or the aforementioned visual perception method.

In the model training method, the visual perception method, the electronic device and the storage medium provided by the embodiments of the present disclosure, the retinal prosthesis uses the spiking recurrent model to predict the spike responses of the ganglion cells. During training of the spiking recurrent model in the retinal prosthesis, firstly, the labels corresponding to the ganglion cells respectively are determined based on the preset ganglion cell response dataset. Then, a plurality of spike signals are obtained as training samples. The spike signals are inputted into the spiking recurrent model one by one. The spike responses of the ganglion cells generated by the spiking recurrent model are obtained, and the loss value is computed based on the spike responses of the ganglion cells, the corresponding labels of the ganglion cells and the preset Poisson loss function. At last, the weight for each layer in the spiking recurrent model is updated based on the loss value computed and the preset time backpropagation function until the updated spiking recurrent model converges. Considering that most of the current retinal prosthesis use a convolutional neural network to predict the responses of the retinal ganglion cells, but the convolutional neural network uses floating-point multiplication for processing, which requires a large amount of computation, consumes a lot of energy and lacks biological similarity, embodiments of the present disclosure convert a sample video into spike signals and iteratively train the spiking recurrent model based on Poisson loss and the time backpropagation function. The model training process is scientific, rigorous and efficient, and the trained spiking recurrent model has low power consumption at work, high prediction accuracy and high biological similarity, effectively improving the visual perception ability of the retinal prosthesis.

In some embodiments, the spiking recurrent model includes a plurality of spike layers and a plurality of recurrent blocks, and updating the weight for each layer in the spiking recurrent model based on the loss value and the preset time backpropagation function includes: updating the weight for each recurrent block based on an output value of each recurrent block, the loss value and the preset time backpropagation function respectively; and updating the weight for each spike layer based on an output value of each spike layer, the loss value, the preset time backpropagation function and a preset gradient proxy function respectively. The spike layers and the recurrent blocks are set to avoid the use of floating-point multiplication and effectively reduce power consumption. Each spike layer has a low spike firing rate, which also greatly contributes to the reduction of power consumption. However, for neurons in the spike layer, the output value of each spike layer is nondifferentiable and cannot be directly backpropagated through time. Therefore, a proxy gradient method is required for processing, that is, a Heaviside function of neurons in the spike layer is replaced with the preset gradient proxy function.

In some embodiments, obtaining the plurality of spike signals as the training samples includes: traversing a plurality of sample videos obtained, and playing a current sample video on a display screen of a preset display apparatus; focusing a preset event camera on the display screen, and obtaining change features of a scene in the current sample video by the event camera; generating and saving spike signals corresponding to the current sample video based on the change features of the scene in the current sample video by a recording apparatus after the current sample video is played; and playing a next sample video on the display screen of the display apparatus after a preset pause duration. To improve the training effect of the spiking recurrent model, the training samples used have to resemble the working conditions of the real human eye, that is, synchronization between playback of the sample videos and collection of the spike signals have to be warranted. The spiking recurrent model trained based on such spike signals can more accurately predict the responses of the retinal ganglion cells, leading to a lower delay of visual perception of the retinal prosthesis.

In some embodiments, communication between the display apparatus and the recording apparatus is maintained through a TCP protocol or IP protocol. The TCP protocol or IP protocol can warrant efficient communication between the display apparatus and the recording apparatus, further improving the synchronization between playback of the sample videos and collection of the spike signals.

In some embodiments, the ganglion cell response dataset is obtained by: repeatedly playing a calibration video containing a first target object several times; recording the responses of the ganglion cells of a second target object who watches the calibration video by an array of electrodes; and generating the ganglion cell response dataset based on the corresponding responses of the ganglion cells during each playback of the calibration video. The determining labels corresponding to the ganglion cells respectively includes: for each of the ganglion cells, taking an average value of the corresponding responses during the several times of playback of the calibration video as the label corresponding to the ganglion cell.

In some embodiments, before obtaining the plurality of spike signals as the training samples, the model training method further includes: reducing a spatial resolution of the event camera to a preset spatial resolution. After obtaining the plurality of spike signals as the training samples, and before inputting the spike signals into the spiking recurrent model, the model training method further includes: filtering the spike signals based on a preset filtering algorithm to obtain filtered spike signals; and decomposing the spike signals into a plurality of spike sequences based on a preset division criterion. The inputting the spike signals into the spiking recurrent model is, specifically, inputting the spike sequences into the spiking recurrent model. To improve the training effect and training efficiency of the spiking recurrent model, a series of preprocessing are performed on the spike signals according to the present disclosure. Reducing the spatial resolution of the event camera may avoid reducing the resolution of each sample video, filtering may effectively remove environmental noise, and decomposing the spike signals into the spike sequences may provide more effective features. The preprocessing may all improve the quality of training samples.

### BRIEF DESCRIPTION OF THE DRAWINGS

One or more embodiments are illustrated through the diagrams in the corresponding drawings. These exemplary descriptions do not constitute a limitation on the embodiments.

FIG. 1 is a schematic diagram of visual degeneration in AMD patients and RP patients;

FIG. 2 is a flowchart of a model training method provided in one embodiment of the present disclosure;

FIG. 3 is a schematic diagram of a model architecture of a spiking recurrent model provided in one embodiment of the present disclosure;

FIG. 4 is a schematic diagram of a model architecture of another spiking recurrent model provided in one embodiment of the present disclosure;

FIG. 5 is a flowchart of obtaining a plurality of spike signals as training samples provided in one embodiment of the present disclosure;

FIG. 6 is a schematic diagram of coordination work of a display apparatus and a recording apparatus provided in one embodiment of the present disclosure;

FIG. 7 is a flowchart of a visual perception method provided in another embodiment of the present disclosure;

FIG. 8 is a schematic structural diagram of an electronic device provided in another embodiment of the present disclosure; and

FIG. 9 is a diagram of comparison in spike sequence and spike count between recorded values of action potential responses of a normal retinal ganglion neuron to a dynamic video and predicted values of a spiking recurrent model provided in another embodiment of the present disclosure.

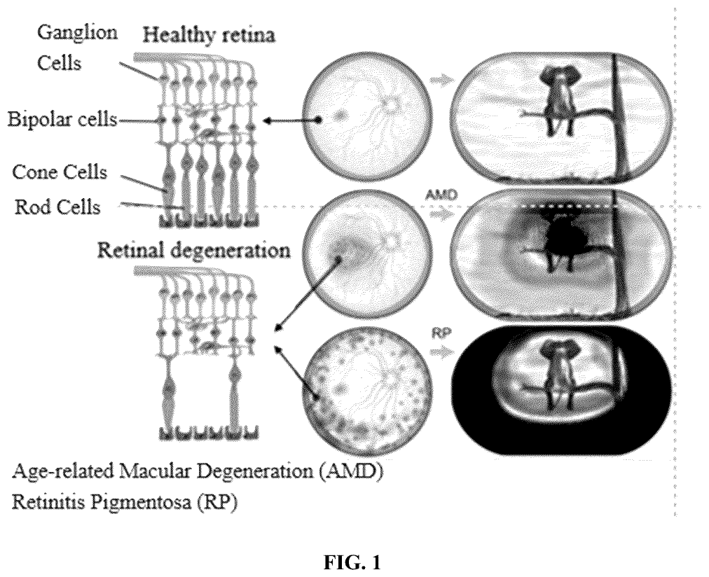

### DETAILED DESCRIPTION OF THE EMBODIMENTS

In order to make the objectives, technical solutions and advantages of embodiments of the present disclosure clearer, the embodiments of the present disclosure will be described in detail below in conjunction with the accompanying drawings. However, those of ordinary skill in the art may understand that in the embodiments of the present disclosure, many technical details are provided to enable readers to better understand the present disclosure. However, even without these technical details and various variations and modifications based on the following embodiments, the technical solutions as claimed in the present disclosure may also be achieved. The division of the following embodiments is for the convenience of description and should not constitute any limitation on the specific implementation of the present disclosure. The embodiments may be combined with and referenced to each other without contradiction.

A retinal prosthesis is a tool that helps AMD patients and RP patients with visual perception, and is essentially required to replace cone and rod cells in functions. The principle of a retinal prosthesis that has been implemented in the industry is to obtain a processed image by collecting signals of an external image using a camera, graying the image, enhancing contrast, extracting edges and merging pixels, and then transmit the processed image to ganglion cells in the last layer of the retina of an implant recipient of the retinal prosthesis through light stimulation for visual perception. However, this type of retinal prosthesis actually only plays a good role in simplifying images and cannot effectively replace cone and rod cells in functions.

The principle of another type of retinal prosthesis that has been implemented in the industry is to capture an external scene using a complementary metal oxide semiconductor (CMOS) image sensor, and predict responses of retinal ganglion cells using a convolutional neural network, thereby replacing damaged cone and rod cells to take effect. However, the processing framework of the convolutional neural network records the external scene using the CMOS image sensor and generates a series of image frames, which can easily lead to data redundancy. In some embodiments, the convolutional neural network uses floating-point multiplication during processing, which results in a large amount of computation during processing and high energy consumption, and actually is not perfectly suitable for retinal prosthesis. Furthermore, the structure of the convolutional neural network and the floating-point multiplication lack biological similarity.

To solve the technical problems of high energy consumption and lack of biological similarity in the aforementioned retinal prosthesis, embodiments of the present disclosure provide a model training method applicable to a spiking recurrent model in a retinal prosthesis. The spiking recurrent model is used for predicting spike responses of ganglion cells and can be applied to an electronic device. The electronic device may be a terminal or a server. In this embodiment and the following embodiments, the electronic device is illustrated using a server as an example. The implementation details of the model training method in this embodiment are specified below. The following content is only for the convenience of understanding the provided implementation details and is not essential for implementing this solution.

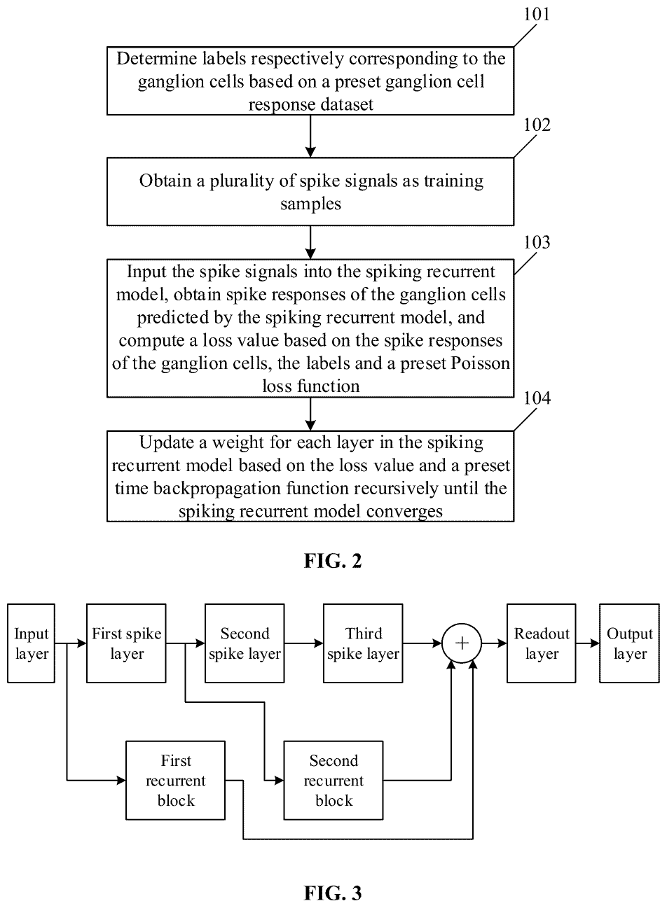

The specific flow of the model training method in this embodiment may be shown in FIG. 2, and include the following operations 101 to 104.

In operation 101, labels corresponding to the ganglion cells respectively are determined based on a preset ganglion cell response dataset.

Specifically, as the spiking recurrent model in the retinal prosthesis is used for predicting the spike responses of the ganglion cells, when the spiking recurrent model is trained, firstly, the ganglion cells are marked with labels as training benchmarks, and the marked labels are used for representing the true responses of the ganglion cells. A server may determine the labels corresponding to the ganglion cells respectively based on a preset ganglion cell response dataset. The preset ganglion cell response dataset records the responses of the ganglion cells to a preset video.

In some embodiments, the ganglion cell response dataset may be a public dataset downloaded from the Internet.

In some embodiments, the server may also first create a ganglion cell response dataset through the following: repeatedly playing a calibration video containing a first target object several times; recording the responses of the ganglion cells of a second target object who watches the calibration video by an array of electrodes; and generating the ganglion cell response dataset based on the corresponding responses of the ganglion cells during each playback of the calibration video. When the labels are determined, for each of the ganglion cells, the server takes an average value of the corresponding responses of the ganglion cells during the several times of playback of the calibration video as the label corresponding to the ganglion cell.

For example, the calibration video may have a duration of 1 minute and a spatial resolution of 360 px×360 px, and the content of the calibration video is a small salamander (the first target object) swimming in the water. When the calibration video is played, the true responses of the ganglion cells of an experimental participant (the second target object) are recorded by the array of electrodes. The calibration video is repeatedly played 30 times, so that 30 true responses are recorded for each ganglion cell. The average value of the 30 true responses is taken for each ganglion cell as the label of the ganglion cell.

In operation 102, a plurality of spike signals are obtained as training samples.

In specific implementation, the spiking recurrent model may receive spike signals as inputs, and the server may obtain a plurality of spike signals as the training samples. For example, an obtained sample video is recorded as spike signals by an event camera, the sample video is played on a display screen, and the event camera is directly focused on the display screen for recording. For another example, a sample video or a sample image is converted into spike signals by software. For another example, spike signals are generated by a random spike generator.

In some embodiments, the event camera may be a dynamic vision sensor (DVS), an asynchronous time-based image sensor (ATIS), a dynamic and active pixel vision sensor (DAVIS), or the like.

In operation 103, the spike signals are inputted into the spiking recurrent model, the spike responses of the ganglion cells generated by the spiking recurrent model are obtained, and a loss value is computed based on the spike responses of the ganglion cells, the corresponding labels and a Poisson loss function.

Specifically, the spiking recurrent model may predict the spike responses of the ganglion cells. Therefore, after the server inputs the spike signals into the spiking recurrent model, the spike responses of the ganglion cells generated by the spiking recurrent model are obtained, and the loss value is computed based on the spike responses of the ganglion cells, the labels corresponding to the ganglion cells and the Poisson loss function.

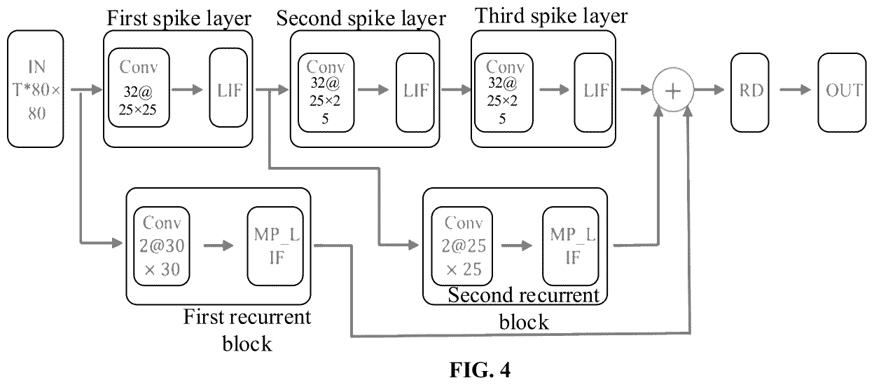

In some embodiments, the model architecture of the spiking recurrent model may be as shown in FIG. 3 and FIG. 4. The model architecture of the spiking recurrent model includes an input layer, a first spike layer, a second spike layer, a third spike layer, a first recurrent block, a second recurrent block, a readout layer and an output layer. The first spike layer, the second spike layer and the third spike layer are sequentially connected. The input layer is connected with the first spike layer and the first recurrent block respectively. The first spike layer is also connected with the second recurrent block. The readout layer is connected with the third spike layer, the first recurrent block and the second recurrent block respectively (the third spike layer, the first recurrent block and the second recurrent block may be connected with the readout layer through an adder). The output layer is connected with the readout layer. As shown in FIG. 4, each spike layer includes a convolutional layer and leaky integrate and fire (LIF) neurons, and each recurrent block includes a convolutional layer and membrane potential LIF (MP_LIF) neurons. The number of LIF neurons in the first spike layer is greater than the number of LIF neurons in the second spike layer, and the number of LIF neurons in the second spike layer is greater than the number of LIF neurons in the third spike layer. The number of MP_LIF neurons in the first recurrent block is equal to the number of MP_LIF neurons in the second recurrent block and the number of LIF neurons in the third spike layer. Such configuration of the spike layers and the recurrent blocks of the spiking recurrent model avoids the use of floating-point multiplication and effectively reduces power consumption. Each spike layer has a low spike firing rate, which also greatly contributes to the reduction of power consumption.

In some embodiments, an initial weight parameter of the convolutional layer in the first spike layer, an initial weight parameter of the convolutional layer in the second spike layer, and an initial weight parameter of the convolutional layer in the third spike layer are all Conv32@25×25. The number of neurons in the first spike layer is 100352, the number of neurons in the second spike layer is 32768, and the number of neurons in the third spike layer is 128. An initial weight parameter of the convolutional layer in the first recurrent block is Conv2@30×30, and an initial weight parameter of the convolutional layer in the second recurrent block is Conv2@25×25. The number of MP_LIF neurons in the first recurrent block and the number of MP_LIF neurons in the second recurrent block are both 128.

In operation 104, a weight for each layer in the spiking recurrent model is updated based on the loss value and a preset time backpropagation function until the updated spiking recurrent model converges.

Specifically, after the loss value is computed, the server may firstly determine whether the spiking recurrent model has been trained to convergence based on the computed loss value and a preset convergence criterion. If the spiking recurrent model has currently been trained to convergence, the next time of training is not needed and a model releasing process is performed. If the spiking recurrent model has not yet been trained to convergence, the server updates the weight for each layer in the spiking recurrent model based on the loss value and the preset time backpropagation function, and then continues to iteratively train the spiking recurrent model until the updated spiking recurrent model converges.

In specific implementation, the spiking recurrent model includes a plurality of spike layers and a plurality of recurrent blocks. The server may update the weight for each recurrent block based on an output value of each of the plurality of the recurrent blocks, the computed loss value and the preset time backpropagation function respectively, and update the weight for each of the plurality of spike layers based on an output value of each of the plurality of spike layers, the computed loss value, the preset time backpropagation function and a preset gradient proxy function respectively. For neurons in each spike layer, the output value of the spike layer is nondifferentiable and cannot be directly backpropagated through time. Therefore, a proxy gradient method is required for processing, that is, a Heaviside function of neurons in the spike layer is replaced with the preset gradient proxy function.

In some embodiments, the server may update the weight for each recurrent block based on the output value of each recurrent block, the computed loss value and the preset time backpropagation function, via the following formula:

$$Δ {w}^{k} = Σ \frac{\partial {L}_{total}}{\partial {o}_{t}^{k}} \frac{\partial {o}_{t}^{k}}{\partial {V}_{t}^{k}} \frac{\partial {V}_{t}^{k}}{\partial {w}^{k}}$$

where w^{k} represents an initial weight of the k-th recurrent block, t represents the time parameter of the time backpropagation function and may be set by a technician based on the actual needs for model training, Vₜ^{k} represents the membrane potential of a MP_LIF neuron in the k-th recurrent block at time t, oₜ^{k} represents the output value of the k-th recurrent block at time t, Lₜₒₜₐₗ represents the loss value, and Δw^{k} represents the updated weight of the k-th recurrent block. For MP_LIF neurons,

$${o}_{t}^{k} = {V}_{t}^{k} = \frac{\partial {o}_{t}^{k}}{\partial {V}_{t}^{k}} = 1 ,$$

which is similar to an artificial neural network (ANN) activation function.

In some embodiments, the server may update the weight of each spike layer based on the output value of each spike layer, the computed loss value, the preset time backpropagation function and the preset gradient proxy function, via the following formulas:

$$Δ {w}^{q} = Σ \frac{\partial {L}_{total}}{\partial {o}_{t}^{q}} \frac{\partial {o}_{t}^{q}}{\partial {V}_{t}^{q}} \frac{\partial {V}_{t}^{q}}{\partial {w}^{q}} \\ \frac{\partial {o}_{t}^{q}}{\partial {V}_{t}^{q}} = {HB}_{1}^{′} ( {V}_{t}^{q} - {V}_{th} ) , {H}_{1} ( x ) = \frac{1}{π} arctan ( π x ) + \frac{1}{2}$$

In the formulas, w^{q} represents the initial weight of the q-th spike layer, Vₜ^{q} represents the membrane potential of a neuron in the q-th spike layer at time t, oₜ^{q} represents the output value of the q-th spike layer at time t, Δw^{q} represents the updated weight of the q-th spike layer, H1(x) represents the preset gradient proxy function, and Vth represents a preset membrane potential threshold.

In the embodiments, the retinal prosthesis uses the spiking recurrent model to predict the spike responses of the ganglion cells. During training of the spiking recurrent model in the retinal prosthesis, firstly, the labels corresponding to the ganglion cells respectively are determined based on the preset ganglion cell response dataset. Then, a plurality of spike signals are obtained as training samples. The spike signals are inputted into the spiking recurrent model one by one, the spike responses of the ganglion cells generated by the spiking recurrent model are obtained, and the loss value is computed based on the spike responses of the ganglion cells, the corresponding labels of the ganglion cells and the preset Poisson loss function. At last, the weight for each layer in the spiking recurrent model is updated based on the loss value computed and the preset time backpropagation function until the updated spiking recurrent model converges. Considering that most of the current retinal prosthesis use a convolutional neural network to predict the responses of the retinal ganglion cells, but the convolutional neural network uses floating-point multiplication for processing, which requires a large amount of computation, consumes a lot of energy and lacks biological similarity, embodiments of the present disclosure convert a sample video into spike signals and iteratively train the spiking recurrent model based on Poisson loss and the time backpropagation function. The model training process is scientific, rigorous and efficient, and the trained spiking recurrent model has low power consumption at work, high prediction accuracy and high biological similarity, effectively improving the visual perception ability of the retinal prosthesis.

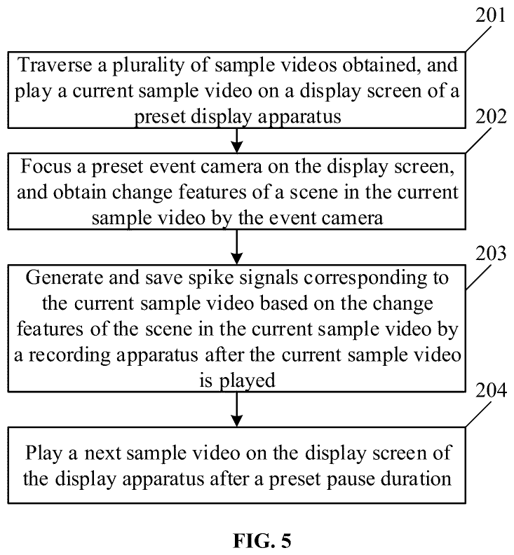

In some embodiments, the server obtaining a plurality of spike signals as training samples may be achieved through sub-operations as shown in FIG. 5, specifically including sub-operations 201 to 204.

In sub-operation 201, a plurality of sample videos obtained are traversed, and a current sample video is played on a display screen of a preset display apparatus.

In sub-operation 202, a preset event camera is focused on the display screen, and change features of a scene in the current sample video are obtained by the event camera.

In specific implementation, the server may traverse the sample videos obtained after obtaining a plurality of sample videos, and play the current sample video on the display screen of the preset display apparatus. The preset event camera is always focused on the display screen of the preset display apparatus, and while the current sample video is played, the event camera obtains the change features of the scene in the current sample video in real time.

In sub-operation 203, a recording apparatus generate and saves spike signals corresponding to the current sample video based on the change features of the scene in the current sample video after the current sample video is played.

In sub-operation 204, a next sample video is played on the display screen of the display apparatus after a preset pause duration.

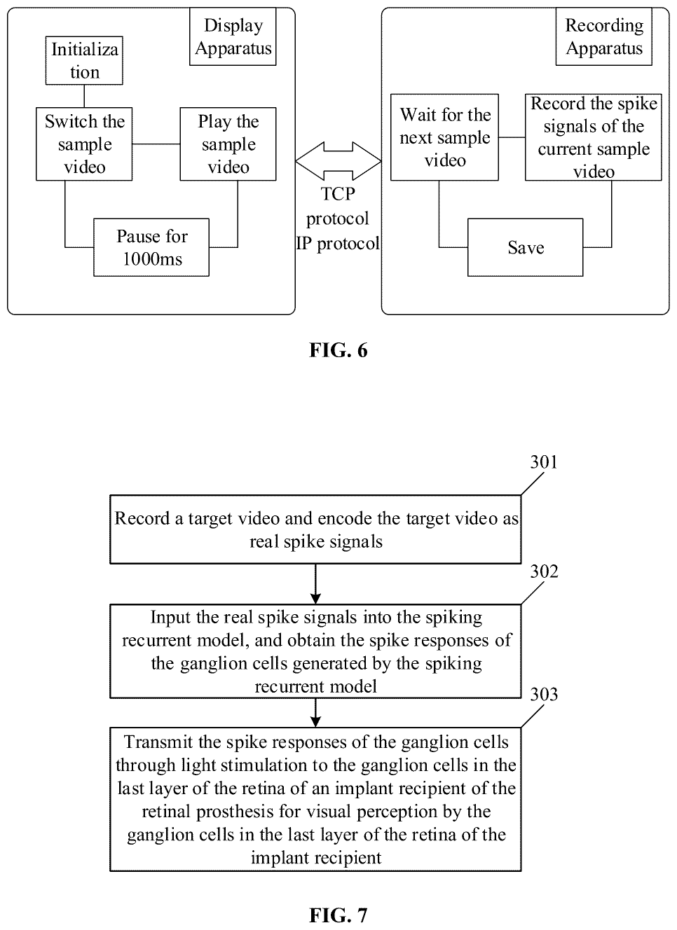

In specific implementation, the process of obtaining the spike signals is jointly completed by the display apparatus, the recording apparatus and the event camera. The display apparatus is responsible for playing and switching the sample videos. The event camera is responsible for focusing on the display screen of the display apparatus to obtain the change features of the scene in the current sample video being played. The recording apparatus is responsible for generating and saving the spike signals corresponding to the current sample video based on the change features of the scene in the current sample video obtained by the event camera. FIG. 6 is a schematic diagram of coordination work of the display apparatus and the recording apparatus. After the current sample video is recorded as spike signals by the recording apparatus and saved, the display apparatus switches to the next sample video and waits for a preset pause duration. After the preset pause duration elapses, the display apparatus plays the previously switched sample video on the display screen, and the recording apparatus starts recording immediately. The preset pause duration may be set to 1000 ms, and communication between the display apparatus and the recording apparatus is maintained through a TCP protocol or IP protocol. The TCP protocol or IP protocol can warrant efficient communication between the display apparatus and the recording apparatus, further improving the synchronization between playback of the sample videos and collection of the spike signals.

In the embodiments, to improve the training effect of the spiking recurrent model, the training samples used have to resemble the working conditions of the real human eye, that is, synchronization between playback of the sample videos and collection of the spike signals have to be warranted. The spiking recurrent model trained based on such spike signals can more accurately predict the responses of the retinal ganglion cells, leading to a lower delay of visual perception of the retinal prosthesis.

In some embodiments, before obtaining a plurality of spike signals as training samples, the server may reduce the spatial resolution of the event camera to a preset spatial resolution. After obtaining the plurality of spike signals as the training samples, and before inputting the spike signals into the spiking recurrent model, the server may filter the spike signals based on a preset filtering algorithm to obtain filtered spike signals, decompose the spike signals into a plurality of spike sequences based on a preset division criterion, and input the spike sequences into the spiking recurrent model. To improve the training effect and training efficiency of the spiking recurrent model, this embodiment performs a series of preprocessing on the spike signals. Reducing the spatial resolution of the event camera may avoid reducing the resolution of each sample video, filtering may effectively remove environmental noise, and decomposing the spike signals into the spike sequences may provide more effective features. The preprocessing may all improve the quality of training samples.

Some embodiments of the present disclosure relates to a visual perception method applicable to a retinal prosthesis. The retinal prosthesis is provided with the spiking recurrent model as described in the aforementioned embodiments, and the spiking recurrent model is used for predicting spike responses of ganglion cells. The implementation details of the visual perception method in this embodiment are specified below. The following content is only for the convenience of understanding the provided implementation details and is not essential for implementing this solution. The specific flow of the visual perception method in this embodiment may, as shown in FIG. 7, include the following operations 301 to 303.

In operation 301, a target video is recorded and encoded as real spike signals.

In operation 302, the real spike signals are inputted into the spiking recurrent model, and the spike responses of the ganglion cells generated by the spiking recurrent model are obtained.

In operation 303, the spike responses of the ganglion cells are transmitted through light stimulation to the ganglion cells in the last layer of the retina of an implant recipient of the retinal prosthesis for visual perception.

In specific implementation, the retinal prosthesis is provided with one or more event cameras. The event camera records the target video by recording a target scene, and the retinal prosthesis encodes the target video as the real spike signals, the real spike signals are inputted into the spiking recurrent model, the spike responses of the ganglion cells generated by the spiking recurrent model are obtained, and at last the spike responses of the ganglion cells are transmitted through light stimulation to the ganglion cells in the last layer of the retina of an implant recipient of the retinal prosthesis for visual perception.

The operation division of the aforementioned methods is only for the purpose of clear description. When implemented, combination into one operation, or splitting of some operation into more operations, as long as the same logical relationship is included, are within the scope of protection of this patent; and adding irrelevant modifications or introducing irrelevant designs to an algorithm or a process, but not changing the core design of the algorithm and the process, is within the scope of protection of this patent.

Some embodiments of the present disclosure relates to an electronic device, as shown in FIG. 8, which includes: at least one processor 401; and a memory 402 communicably connected with the at least one processor 401, for storing instructions executable by the at least one processor 401. Execution of the instructions by the at least one processor 401 causes the at least one processor 401 to implement the model training method in the aforementioned embodiment, or to implement the visual perception method in the aforementioned embodiment.

The memory and the processor are connected by buses, and the buses may include any number of interconnected buses and bridges. The buses connect various circuits of one or more processors and the memory together. The buses may also connect various other circuits of, for example, a peripheral device, a voltage regulator and a power management circuit together, which is well-known in the art and therefore will not be further described herein. A bus interface provides an interface between a bus and a transceiver. The transceiver may be a single component or a plurality of components, for example, a plurality of receivers and transmitters, providing a unit for communication with various other apparatuses on a transmission medium. Data processed by the processor is transmitted on a wireless medium through an antenna, and further, the antenna receives and transmits data to the processor.

The processor is responsible for managing the buses and usual processing, and may also provide various functions, including timing, peripheral interfaces, voltage regulation, power management and other control functions. The memory may be configured for storing data used by the processor during operation.

Some embodiments of the present disclosure relates to a computer-readable storage medium on which a computer program is stored. The computer program is executed by a processor to implement the aforementioned method embodiments.

That is, those skilled in the art may understand that implementation of all or part of the operations of the methods in the aforementioned embodiments may be completed by instructing relevant hardware through a program. The program is stored on a storage medium and includes a plurality of instructions for enabling a device (which may be a microcontroller, a chip and the like) or a processor to execute all or part of the operations of the methods in the embodiments of the present disclosure. The storage medium includes: any medium that can store program code, such as a USB flash drive, a removable hard disk, a read-only memory (ROM), a random access memory (RAM), a magnetic disk, or an optical disc.

The performance verification results of the spiking recurrent model will be introduced in another embodiment below.

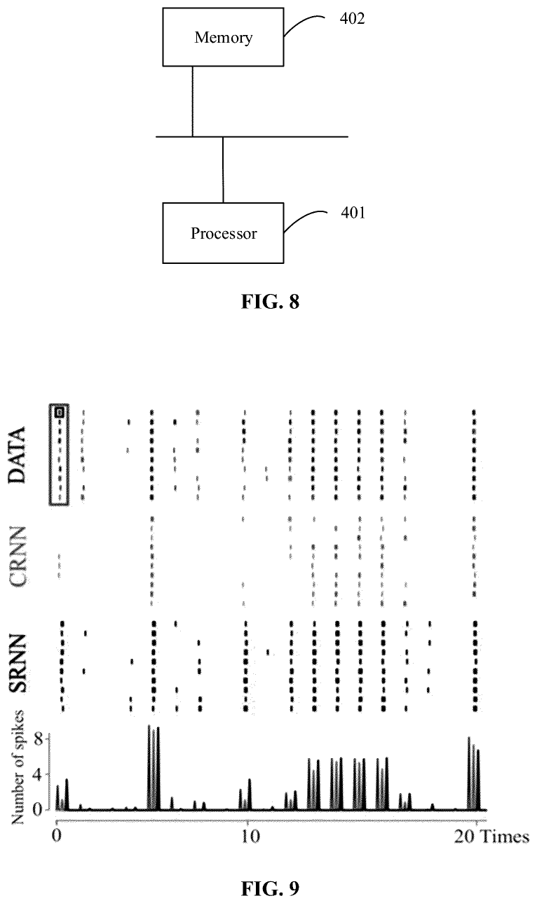

FIG. 9 shows comparison in spike sequences and spike count between recorded values of action potential responses of a normal retinal ganglion to a dynamic video and predicted values of the spiking recurrent model. Data in FIG. 9 is the recorded action potential responses of normal retinal ganglion cells to an external dynamic scene. CRNN in FIG. 9 represents the fitting effect of a convolutional neural network on ganglion cells. SRNN in FIG. 9 represents the fitting effect of a spiking recurrent processing algorithm on the response pattern of ganglion cells. The spike sequences inside the rectangle are generated by the number of action potentials predicted based on an SRNN model using Poisson distribution. This spike generation method is a scientific method for predicting the time and number of spike occurrences within a continuous time period. In some embodiments, the spike sequences inside the rectangle are the results obtained by repeating the above process ten times, and the repetitive process may effectively avoid randomness. Other spike sequences are obtained using the same method. By comparing the spike sequences and the spike count, it can be seen that the spike sequences of the SRNN model can well fit the response behavior of neurons with a higher spike firing rate, slightly better than those of the CRNN model. Both models have limited performance on neurons with a lower spike firing rate. The bottom column displays a comparison in the spike count, demonstrating a high degree of similarity between the predicted values of the SRNN model and the experimental recorded values. In contrast, the performance of the CRNN model is poor in fitting neurons with a lower spike firing rate. In summary, the spiking recurrent model may well replace the processing function of a retina and be applied in visual prosthesis.

Those of ordinary skill in the art may understand that the aforementioned embodiments are specific embodiments for implementing the present disclosure, but in practical applications, various changes may be made in form and details without deviating from the spirit and scope of the present disclosure.

What is claimed is:

1. A model training method applicable to a spiking recurrent model in a retinal prosthesis, the spiking recurrent model being used for predicting spike responses of ganglion cells, and the model training method comprising:
determining labels respectively corresponding to the ganglion cells based on a preset ganglion cell response dataset;
obtaining a plurality of spike signals as training samples;
inputting the plurality of spike signals into the spiking recurrent model, obtaining spike responses of the ganglion cells predicted by the spiking recurrent model, and computing a loss value based on the spike responses of the ganglion cells, the labels and a preset Poisson loss function; and
updating a weight for each layer in the spiking recurrent model based on the loss value and a preset time backpropagation function recursively until the spiking recurrent model converges.

2. The model training method according to claim 1, wherein the spiking recurrent model includes a plurality of spike layers and a plurality of recurrent blocks, and updating the weight for each layer in the spiking recurrent model based on the loss value and the preset time backpropagation function includes:
updating the weight for each of the plurality of recurrent blocks based on an output value of each of the plurality of recurrent blocks, the loss value and the preset time backpropagation function respectively; and
updating the weight for each of the plurality of spike layers based on an output value of each of the plurality of spike layers, the loss value, the preset time backpropagation function and a preset gradient proxy function respectively.

3. The model training method according to claim 2, wherein the weight of each of the plurality of recurrent blocks is updated based on the output value of each of the plurality of recurrent blocks, the loss value and the preset time backpropagation function by the following formula:
$$Δ {w}^{k} = Σ \frac{\partial {L}_{total}}{\partial {o}_{t}^{k}} \frac{\partial {o}_{t}^{k}}{\partial {V}_{t}^{k}} \frac{\partial {V}_{t}^{k}}{\partial {w}^{k}}$$
wherein w^{k} represents an initial weight of a k-th recurrent block, t represents a time parameter of the time backpropagation function, Vₜ^{k} represents a membrane potential of a membrane potential neuron in the k-th recurrent block at time t, oₜ^{k} represents an output value of the k-th recurrent block at time t, Lₜₒₜₐₗ represents the loss value, and Δw^{k} represents an updated weight of the k-th recurrent block;
the weight for each of the plurality of spike layers is updated based on the output value of each of the plurality of spike layers, the loss value, the preset time backpropagation function and the preset gradient proxy function by the following formulas:
$$Δ {w}^{q} = Σ \frac{\partial {L}_{total}}{\partial {o}_{t}^{q}} \frac{\partial {o}_{t}^{q}}{\partial {V}_{t}^{q}} \frac{\partial {V}_{t}^{q}}{\partial {w}^{q}} \\ \frac{\partial {o}_{t}^{q}}{\partial {V}_{t}^{q}} = {HB}_{1}^{′} ( {V}_{t}^{q} - {V}_{th} ) , {H}_{1} ( x ) = \frac{1}{π} arctan ( π x ) + \frac{1}{2}$$
wherein w^{q} represents an initial weight of a q-th spike layer, Vₜ^{q} represents a membrane potential of a neuron in the q-th spike layer at time t, oₜ^{q} represents an output value of the q-th spike layer at time t, Δw^{q} represents an updated weight of the q-th spike layer, H₁(x) represents the preset gradient proxy function, and Vₜₕ represents a preset membrane potential threshold.

4. The model training method according to claim 1, wherein obtaining the plurality of spike signals as the training samples includes:
traversing a plurality of sample videos obtained, and playing a current sample video on a display screen of a preset display apparatus;
focusing a preset event camera on the display screen, and obtaining change features of a scene in the current sample video by the event camera;
generating and saving spike signals corresponding to the current sample video based on the change features of the scene in the current sample video by a recording apparatus after the current sample video is played; and
playing a next sample video on the display screen of the display apparatus after a preset pause duration.

5. The model training method according to claim 2, wherein obtaining the plurality of spike signals as the training samples includes:
traversing a plurality of sample videos obtained, and playing a current sample video on a display screen of a preset display apparatus;
focusing a preset event camera on the display screen, and obtaining change features of a scene in the current sample video by the event camera;
generating and saving spike signals corresponding to the current sample video based on the change features of the scene in the current sample video by a recording apparatus after the current sample video is played; and
playing a next sample video on the display screen of the display apparatus after a preset pause duration.

6. The model training method according to claim 3, wherein obtaining the plurality of spike signals as the training samples includes:
traversing a plurality of sample videos obtained, and playing a current sample video on a display screen of a preset display apparatus;
focusing a preset event camera on the display screen, and obtaining change features of a scene in the current sample video by the event camera;
generating and saving spike signals corresponding to the current sample video based on the change features of the scene in the current sample video by a recording apparatus after the current sample video is played; and
playing a next sample video on the display screen of the display apparatus after a preset pause duration.

7. The model training method according to claim 4, wherein communication between the display apparatus and the recording apparatus is maintained through a TCP protocol or IP protocol.

8. The model training method according to claim 1, wherein the ganglion cell response dataset is obtained by:
repeatedly playing a calibration video containing a first target object several times;
recording the responses of the ganglion cells of a second target object who watches the calibration video by an array of electrodes; and
generating the ganglion cell response dataset based on the corresponding responses of the ganglion cells during each playback of the calibration video;
wherein determining the labels corresponding to the ganglion cells respectively includes:
for each of the ganglion cells, taking an average value of the corresponding responses during the several times of playback of the calibration video as a label corresponding to a respective one of the ganglion cells.

9. The model training method according to claim 2, wherein the ganglion cell response dataset is obtained by:
repeatedly playing a calibration video containing a first target object several times;
recording the responses of the ganglion cells of a second target object who watches the calibration video by an array of electrodes; and
generating the ganglion cell response dataset based on the corresponding responses of the ganglion cells during each playback of the calibration video;
wherein determining the labels corresponding to the ganglion cells respectively includes:
for each of the ganglion cells, taking an average value of the corresponding responses during the several times of playback of the calibration video as a label corresponding to a respective one of the ganglion cells.

10. The model training method according to claim 3, wherein the ganglion cell response dataset is obtained by:
repeatedly playing a calibration video containing a first target object several times;
recording the responses of the ganglion cells of a second target object who watches the calibration video by an array of electrodes; and
generating the ganglion cell response dataset based on the corresponding responses of the ganglion cells during each playback of the calibration video;
wherein determining the labels corresponding to the ganglion cells respectively includes:
for each of the ganglion cells, taking an average value of the corresponding responses during the several times of playback of the calibration video as a label corresponding to a respective one of the ganglion cells.

11. The model training method according to claim 4, wherein before obtaining the plurality of spike signals as the training samples, the method further includes:
reducing a spatial resolution of the event camera to a preset spatial resolution; and
after obtaining the plurality of spike signals as the training samples, and before inputting the plurality of spike signals into the spiking recurrent model, the model training method further includes:
filtering the plurality of spike signals based on a preset filtering algorithm to obtain filtered spike signals; and
decomposing the plurality of spike signals into a plurality of spike sequences based on a preset division criterion; and
the inputting the plurality of spike signals into the spiking recurrent model is inputting the plurality of spike sequences into the spiking recurrent model.

12. A visual perception method applicable to a retinal prosthesis, the retinal prosthesis being provided with the spiking recurrent model according to claim 1, the spiking recurrent model being used for predicting spike responses of ganglion cells, and the visual perception method comprising:
recording a target video and encoding the target video as real spike signals;
inputting the real spike signals into the spiking recurrent model, and obtaining the spike responses of the ganglion cells generated by the spiking recurrent model; and
transmitting the spike responses of the ganglion cells through light stimulation to the ganglion cells in a last layer of the retina of an implant recipient of the retinal prosthesis for visual perception.

13. An electronic device, comprising:
at least one processor; and,
memory communicably connected with the at least one processor for storing instructions executable by the at least one processor, wherein execution of the instructions by the at least one processor causes the at least one processor to implement the model training method according to claim 1.

14. A non-transitory computer-readable storage medium on which a computer program is stored, wherein the computer program is executed by a processor to implement the model training method according to claim 1.
